(19) United States
(12) Patent Application Publication
(10) Pub. No.: US 2011/0244569 A1
(43) Pub. Date: Oct. 6, 2011

(54) METHOD OF INDUCING PROLIFERATION AND/OR DIFFERENTIATION OF NEURAL PRECURSOR CELLS BY INTRODUCING PROLACTIN OR WNT3A TO ACTIVATE LATENT NEURAL PRECURSOR CELLS

(76) Inventors: Tara Louise Walker, Queensland (AU); Perry Francls Bartlett, Queensland (AU)

(21) Appl. No.: 12/992,500

(22) PCT Filed: May 13, 2009
(86) PCT No.: PCT/AU2009/000594

§ 371 Date: Jun. 23, 2011

(30) Foreign Application Priority Data
May 13, 2008 (AU) 2008902357

(51) Int. Cl. C12N 5/079

(52) U.S. Cl. 435/375

(57) ABSTRACT

A method of inducing proliferation and/or differentiation of a hippocampal cell population activating a latent neural precursor cell, enriching a cell population for neural precursor cells and treating neurodegenerative diseases and/or repopulating a damaged hippocampus by introducing prolactin or Wnt3a so as to activate a latent neural precursor cell population.

### TECHNICAL FIELD

The present invention relates to factors which are able to activate a hippocampal neural precursor cell population.

### BACKGROUND ART

Many neurological diseases such as dementia, including Alzheimer's disease, stroke, depression, Parkinson's disease and motor neuron disease are associated with a reduction in the number of neurons. The decline in number of neurons may be rapid, as in the case of stroke, or slower, as in the case of Alzheimer's disease.

After heart disease and cancer, stroke is the third leading cause of death in western industrialized countries and the major cause of severe, long-term disability in adults with 56% of people following a stroke suffering from a severe or profound disability. There are over 20 million stroke survivors worldwide. Over 40,000 stroke events occur in Australia each year—one every 12 minutes, one every 45 seconds in the USA. This ailment represents an economic burden estimated to be $45 billion a year in the US alone and is expected to rise significantly. A significant factor contributing to this trend is the increased susceptibility to stroke among the elderly.

Alzheimer's disease is the most common dementia occurring in the elderly, affecting about 10% of people above 65 years and 40% above 80 years. Alzheimer's disease is predicted to afflict up to 16 million people by the middle of this century unless a cure or prevention is found in the United States alone. 50-75% of dementia is estimated to be caused by Alzheimer's disease. The prevalence of Alzheimer's disease is slightly higher in women than in men, but almost twice as many women live with dementia because of their longer life expectancy.

Alzheimer's disease is a progressive neurodegenerative disease characterized by memory loss and general cognitive and behavioural decline. Alzheimer's disease is commonly associated with a non-cognitive symptomatology including depression. Histologically, Alzheimer's disease is defined by the presence in post-mortem human brain specimens of amyloid neuritic plaques, the formation of neurofibrillary tangles and degeneration of the cholinergic neurons.

Parkinson's disease is associated with the destruction of neurons, but the damage is restricted to the dopamine-producing cells in the substantia nigra (part of the basal ganglia). The most common symptoms of Parkinson's disease are tremor, rigidity and difficulty initiating movement. In the US alone, some one million patients are affected and 50,000 new patients are added annually.

Motor Neuron Disease is the name given to a group of diseases in which nerve cells that control the muscles degenerate and die. It is rarely diagnosed in people less than 30 years of age. In Australia, there are around 400 new cases of Motor Neuron Disease each year. There is no effective method of treatment and the disease is generally fatal within 1-5 years of diagnosis. More than one person dies of Motor Neuron Disease each day in Australia.

There are several regions in the brain where stem cells are known to exist, including the sub-ventricular zone and the olfactory bulb. It is thought that the stem cells in these areas are already working at maximum capacity to generate new neurons for general “self-maintenance”. Additionally, neurogenesis occurs in the subgranular cell layer of the adult hippocampal dentate gyrus and has important consequences in learning and memory (Shors et al., 2001). A number of environmental and behavioural stimuli have been shown to enhance hippocampal neurogenesis and this is thought to be mediated through synaptic activity (van Praag et al 1999; Brown et al., 2003; Santarelli et al., 2003; McEwen, 1994; Arvidsson et al., 1994). Since large numbers of hippocampal cells are generated throughout the lifetime of an animal it is predicted that a stem cell is most likely responsible. Surprisingly, however, the precursors identified so far that generate these cells have limited self-renewal (Bull and Bartlett 2005; Seaberg and van der Kooy et al., 2002). Despite the limited capacity for self-renewal of the cells so far identified, it has been shown that excitation caused by applying depolarizing levels of extracellular potassium mimics the effects of stably increased activity, as would occur in an active neural network, and leads to an increase in neuron production from hippocampal adult neural progenitor cells (Deisseroth et al., 2004).

In co-pending International Patent Application No. PCT/AU2008/000511, the contents of which are incorporated herein by reference, the present inventors described a population of neural stem cells derived from the adult hippocampus, and also apparently located in the olfactory bulb, cortex, cerebellum or spinal cord, which may be activated by depolarization of the cell membrane. Although the expression of the protein Doublecortin (DCX) is ordinarily associated with commitment of cells to the neuronal lineage, the cells which respond to depolarization are DCX^{-ve}. These cells have additionally been shown to bind the lectin peanut agglutinin (PNA) and so are considered to be PNA^{+ve}. This neural precursor cell population is additional to the known populations such as those in the SVZ and the olfactory bulb, and different in character.

United States Patent Application No. 2003/0054998 (Shingo and Weiss) describes a method of increasing neural stem cell numbers or neurogenesis by using prolactin. The entire sub-ventricular forebrain was collected from mice infused with prolactin. The results show that infusion of prolactin increased BrdU-labeled cells in the forebrain SVZ. Therefore, prolactin was shown to stimulate cell proliferation in the subventricular zone, but no effect in other areas of the brain was noted. To determine the effect of prolactin on cultured neural stem cells derived from the SVZ of adult mice were incubated for 7 days in the presence of EGF alone, or EGF plus prolactin, in addition to basal media. While prolactin alone did not significantly increase the number of neurospheres in vitro, the results indicate that prolactin is capable of potentiating the effect of EGF to increase the number of neurospheres, but only in a culture of neural stem cells derived from the SVZ.

United States Patent Application No. 2008/0213892 (Nusse et al) describes an in vitro method for expanding neural progenitor or stem cells in a medium comprising application of purified Wnt protein. The inventors indicate that they “showed that there is a small subpopulation of cells lining the neurogenic zone (SVZ) in the developing mouse brain that is Wnt-responsive.” Thus the population of stem cells for which Wnt activity is asserted is the conventional population derived from the SVZ, and is not the population identified in PCT/AU2008/000511.

### SUMMARY OF THE INVENTION

The present inventors have now identified factors whose production is increased as a result of membrane depolarization of neural cell populations and which themselves activate the neural precursor cell population. Surprisingly prolactin and Wnt3a have been found to activate a latent neural precursor cell in a hippocampal cell population as well as the previously known and distinct population of neural stem cells derived from the SVZ, but this has been hitherto unrecognised. Additionally, this latent cell population may be characterised as empty spiracles homolog 1 positive (Emx1^{+ve}). Further, this latent cell population may be characterised as glial fibrilliary acidic protein positive (GFAP^{+ve}).

Accordingly, in one aspect, the present invention provides a method of inducing proliferation and/or differentiation of a hippocampal cell population, comprising:

(1) providing a hippocampal cell population comprising a latent neural precursor cell;

(2) introducing the hippocampal cell population to a culture medium; and

(3) introducing prolactin or Wnt3a to the culture medium so as to activate the latent neural precursor cell.

Accordingly, in a further aspect, the present invention provides a method of activating a latent neural precursor cell contained within a hippocampal cell population, comprising:

(1) providing a hippocampal cell population comprising a latent neural precursor cell;

(2) introducing the hippocampal cell population to a culture medium; and

(3) activating the neural precursor cell by introducing prolactin or Wnt3a to the culture medium.

In an embodiment the culture medium is a neurosphere-forming culture.

In a still further aspect, the present invention provides a method of establishing a neural cell population enriched for neural precursor cells, comprising:

(1) providing a hippocampal cell population comprising a latent neural precursor cell;

(2) introducing the hippocampal cell population to a neurosphere-forming culture medium;

(3) introducing prolactin or Wnt3a to the culture medium so as to activate the latent neural precursor cell; and

(4) selecting cells which demonstrate the property of self-renewal and multipotency.

It has been observed that the cells most likely to demonstrate the properties of self-renewal and multipotency are derived from neurospheres which are large in diameter, particularly those neurospheres larger in diameter than 110 μm and more particularly larger in diameter than 250 μm. Accordingly, in an embodiment neurospheres which have been subjected to activation and grow larger in diameter than 110 μm and preferably larger in diameter than 250 μm are selected and prima facie identified as yielding a cell population in accordance with the invention for subsequent verification.

The method of the invention may further comprise inducing differentiation and proliferation.

The expanded and activated precursor cell population, or the progeny thereof following differentiation and proliferation, is likely to be useful in the treatment of neurodegenerative diseases to reverse the decline in the number of neurons characteristic of those diseases.

In an embodiment, the progeny of the precursor cell population is introduced to the animal by transplantation involving implantation of the cells into the animal.

In particular, the discovery of factors which activate a latent neural precursor cell population opens the possibility that the in vivo population can be stimulated to proliferate and differentiate.

Accordingly, in a still further aspect the present invention provides a method of treating a neurodegenerative disease or repopulating a damaged hippocampus after brain injury comprising administering prolactin or Wnt3a to an animal in need of such treatment so as to activate in vivo a latent neural precursor cell population.

In a still further aspect there is provided the use of prolactin or Wnt3a in the manufacture of a medicament for the treatment of a neurodegenerative disease or repopulation of a damaged hippocampus after brain injury wherein the medicament activates in vivo a latent neural precursor cell population.

In a still further aspect there is provided the use of prolactin or Wnt3a to activate in vivo a latent neural precursor cell population for the treatment of a neurodegenerative disease or repopulation of a damaged hippocampus after brain injury.

In an embodiment prolactin or Wnt3a is administered to the hippocampus in order to activate the latent neural precursor cell population.

In an embodiment the neurodegenerative disease is selected from the group consisting of Alzheimer's disease, Parkinson's disease and motor neuron disease.

In an embodiment repopulation of a damaged hippocampus may be undertaken after brain injuries such as stroke or ischemia leading to some degree of long-range connections and improved functional recovery.

### BRIEF DESCRIPTION OF THE FIGURES

FIG. 1 shows potassium-induced activation of a neural precursor cell population from the hippocampus and failure of a population from the sub-ventricular zone (SVZ) to activate it (A) graph showing degree of activation of cells in the hippocampus and SVZ in the presence and absence of potassium chloride (B) graph showing neurosphere diameter for hippocampal cells in the presence and absence of potassium chloride (C) photograph comparing an average neurosphere to one of the large neurospheres produced under high potassium conditions (D) graph demonstrating ability of potassium-activated cells to be passaged repeatedly.

FIG. 2 is a graph showing the influence of potassium chloride concentration on cell activation.

FIG. 3 is a graph demonstrating activation by barium chloride compared to a control of sodium chloride.

FIG. 4 is a graph showing inducement of activity with potassium chloride in the presence and absence of the L-type calcium channel antagonist, nicardipine.

FIG. 5 demonstrates results for the potassium-activated cell population (A) a DCX^{-ve} population is expanded by potassium induction (B) potassium-induced activation with increasing age of the hippocampus.

FIG. 6 demonstrates the inability to expand the cell population in the SVZ.

FIG. 7 demonstrates expansion of a cell population (A) in the olfactory bulb (B) in the cortex and (C) in the cerebellum.

FIG. 8 shows results for in vivo models (A) demonstration of activation by induction of seizures in mice with injections of pilocarpine hydrochloride (B) demonstration of activation in Huntington's disease mice (C) activation with increasing age in wild type mice using potassium chloride and (D) equivalent experiment in Huntington's disease mice demonstrating that after 33 weeks potassium chloride could not further increase depolarization in vitro.

FIG. 9 is a plot of activation in cells derived from the spinal cord demonstrating activation with KCl.

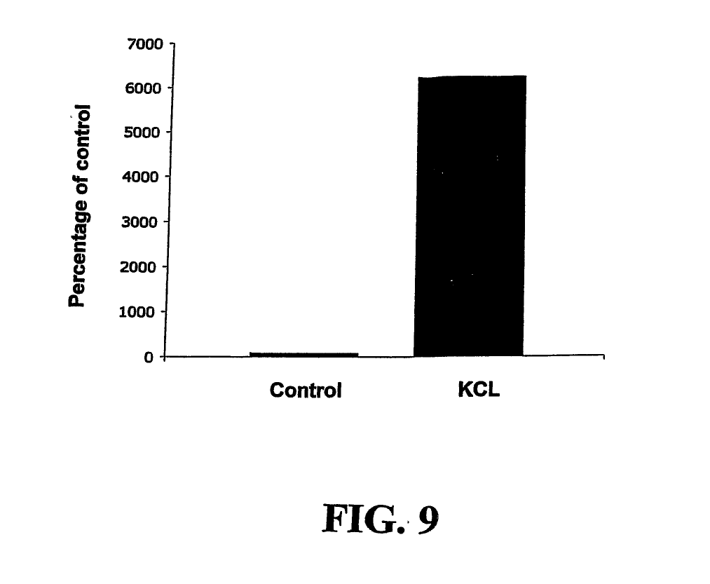

FIG. 10 is a graph demonstrating activation of hippocampal cells with prolactin (PRL) but not cells of the SVZ.

FIG. 11 is a graph demonstrating activation of hippocampal cells with Wnt3a.

FIG. 12 is a plot of activation in an 18 month hippocampal population of cells demonstrating activation by Wnt3a and prolactin (PRL) compared to a control.

FIG. 13 is a plot showing neurosphere numbers in the Hippocampus and the SVZ for mice that are wild-type or heterozygous for an inactivating mutation in the prolactin (PRL) receptor.

FIG. 14 is a plot showing neurosphere numbers for wild-type, heterozygotes and prolactin knock-out mice.

### DETAILED DESCRIPTION OF THE INVENTION

Depolarization of the membrane may be employed to activate a neural cell population as described in International Patent Application No. PCT/AU2008/000511. It has now been found that activation of a precursor cell population, including a stem cell population, may be achieved by introducing a factor whose production is increased as a result of membrane depolarization. While not wishing to be bound by theory it is believed that depolarization induces opening of L-type voltage-gated calcium channels and this in turn induces increased production of these factors. The L-type calcium channel typically opens in mature neurons in response to the depolarization provided by excitatory synaptic inputs. Thus excitation may be achieved through electrical signalling, but also through altering ionic concentrations in the environment of this cell or by contacting the cell with chemical agents that are specific modulators of activity of the calcium channel and serve to activate the channel. Voltages-sensitive calcium channels play an important role in regulating hormone and neurotransmitter release, muscle contraction, and a large number of other cellular functions. A modulator of activity of the calcium channel interacts with it, such as by binding to it, to alter the amount or duration of the biological activity of the calcium channel. An agonist of the calcium channel is a molecule that activates the calcium channel and so triggers an influx of calcium to the cell. The entry of calcium into a cell such as a mature neuron activates a signalling pathway and thus specifies a cellular response to calcium. Agonists of the calcium channel and the effects induced are known in the art. For example, they are sensitive to dihydropyridine agonists and antagonists. Antagonists of calcium channels comprise three different chemical classes: the dihydropyridine antagonists such as nifedipine, amlodipine, nitrendipine, nisoldipine and nicardipine; phenylalkylamines such as verapamil and benzothiazepines such as diltiazem. Modulators of activity can be proteins, carbohydrates, antibodies or low molecular weight molecules.

In addition to chemical modulation, activation can be achieved by electrical stimulation. This may be achieved by direct electrical excitation, for example, through electroconvulsive therapy, transcranial magnetic stimulation, deep brain stimulation or vagal nerve stimulation, or by altering the ionic strength of the environment of the cell. In an embodiment the ionic strength of the environment of the cell is increased by between 2 and 10 times, preferably between 4 and 6 times, most preferably about 5 times. In an embodiment, activation is achieved through contacting the cell with a potassium salt in excess of physiological concentration, for example, 10 to 40 mM potassium chloride, preferably 12 to 30 mM potassium chloride, most preferably 15 mM potassium chloride. In an alternative embodiment activation can be achieved through contacting the cell with a barium salt such as barium chloride.

While not wishing to be bound by theory, it is believed that membrane depolarization induces opening of L-type voltage-gated calcium channels. These ion channels are involved in numerous signalling pathways, and the present inventors have observed significant upregulation of the following:

TABLE 1
Genes that are upregulated in activated cell population

| Gene Symbol | Gene name |
|---|---|
| Slc5a1 | solute carrier family 5 (sodium/glucose cotransporter), |
| | member l |
| Wnt3a | wingless-related MMTV integration site 3A |
| Ahnak | AHNAK nucleoprotein (desmoyokin) |
| Tcfap2c | transcription factor AP-2, gamma |
| Setdb1 | SET domain, bifurcated 1 |
| Igf2bp3 | insulin-like growth factor 2 mRNA binding protein 3 |
| Iapp | islet amyloid polypeptide |
| Hes2 | hairy and enhancer of split 2 (Drosophila) |
| Gng2 | guanine nucleotide binding protein (G protein), |
| | gamma 2 subunit |
| Cylcl | cylicin, basic protein of sperm head cytoskeleton 1 |
| EG434726 /// | ferritin, heavy polypeptide-like 17 /// predicted gene, |
| EG434727 /// | EG434726 /// predicted gene, EG434727 /// predicted |
| EG434728 /// | gene, EG434728 |
| Fthll7 | |
| Igk-V28///I | immunoglobulin kappa chain variable 28 (V28) /// |
| | immunoglobulin kappa light chain 17-1A |
| Enox2 | ecto-NOX disulfide-thiol exchanger 2 |
| Aurkb | aurora kinase B |
| Zkscan14 | zinc finger with KRAB and SCAN domains 14 |
| Serpina5 | serine (or cysteine) peptidase inhibitor, clade A, |
| | member 5 |
| Crkrs | Cdc2-related kinase, arginine/serine-rich |
| Ntf3 | neurotrophin 3 |
| Hs1bp3 | HCLS1 binding protein 3 |
| Foxml | forkhead box M1 |
| Itgav | integrin alpha V |
| Eif4g1 | eukaryotic translation initiation factor 4, gamma 1 |
| Myb | myeloblastosis oncogene |
| Rabgap1 | RAB GTPase activating protein 1 |
| Rcbtb2 | regulator of chromosome condensation (RCC1) and |
| | BTB (POZ) domain containing protein 2 |
| D1Ertd83e | DNA segment, Chr 1, ERATO Doi 83, expressed |
| Col17a1 | procollagen, type XVII, alpha 1 |
| Pwcr1 | Prader-Willi chromosome region 1 homolog (human) |
| Chrna5 | cholinergic receptor, nicotinic, alpha polypeptide 5 |
| Adh4 | alcohol dehydrogenase 4 (class II), pi polypeptide |
| Serpinb9c | serine (or cysteine) peptidase inhibitor, clade B, |
| | member 9c |
| Ncapg | on-SMC condensin I complex, subunit G |
| LOC639910 | hypothetical protein LOC639910 |
| Slc22a3 | solute carrier family 22 (organic cation transporter), |
| | member 3 |
| Lrp6 | low density lipoprotein receptor-related protein 6 |
| Gsh1 | genomic screened homeo box 1 |
| Dbf4 | DBF4 homolog (S. cerevisiae) |
| Prrg2 | proline-rich Gla (G-carboxyglutamic acid) polypeptide 2 |
| LOC669660 /// | PDZ and LIM domain 5 /// similar to PDZ and LIM |
| | domain |
| Pdlim5 | protein 5 (Enigma homolog) (Enigma-like PDZ and LIM |
| | domains protein) |
| Hbb-bh1 | hemoglobin Z, beta-like embryonic chain |
| Abcc9 | ATP-binding cassette, sub-family C (CFTR/MRP), |
| | member 9 |
| Gata6 | GATA binding protein 6 |
| H2-D1 | histocompatibility 2, D region locus 1 |
| Nlrp4c | NLR family, pyrin domain containing 4C |
| Fbxo15 | F-box protein 15 |
| Sim1 | single-minded homolog 1 (Drosophila) |
| Bcl2a1a /// | B-cell leukemia/lymphoma 2 related protein Ala /// B-cell |
| Bcl2a1b /// | leukemia/lymphoma 2 related protein A1b /// B-cell |
| Bcl2a1c /// | leukemia/lymphoma 2 related protein A1c /// B-cell |
| Bcl2a1d | leukemia/lymphoma 2 related protein A1d |
| Rab17 | RAB17, member RAS oncogene family |
| Prl | prolactin |
| Rarres2 | retinoic acid receptor responder (tazarotene induced) 2 |
| 2900060N18Rik | RIKEN cDNA 2900060N18 gene |
| Lin7b | lin-7 homolog B (C. elegans) |
| Clgn | calmegin |
| Ugt8a | UDP galactosyltransferase 8A |
| B3galt1 | UDP-Gal: betaGlcNAc beta 1,3-galactosyltransferase, |
| | polypeptide 1 |
| Pof1b | premature ovarian failure 1B |
| Atp6v0a1 | ATPase, H+ transporting, lysosomal V0 subunit A1 |
| Bves | blood vessel epicardial substance |
| Itga4 | integrin alpha 4 |
| Iqgap1 | IQ motif containing GTPase activating protein l |
| Olfr64///OI | olfactory receptor 64 /// olfactory receptor 66 |
| Tbc1d14 | TBC1 domain family, member 14 |
| Tpt1 | tumor protein, translationally-controlled 1 |
| Rhov | ras homolog gene family, member V |
| Brwdl | bromodomain and WD repeat domain containing 1 |
| Krtap15 | keratin associated protein 15 |
| Atf7 | activating transcription factor 7 |
| Scgb1a1 | secretoglobin, family 1A, member 1 |
| | (uteroglobin) /// cDNA sequence U46068 |
| Gprin1 | G protein-regulated inducer of neurite outgrowth 1 |
| Eras | ES cell-expressed Ras |
| Alx3 | aristaless 3 |
| Avprlb | arginine vasopressin receptor lB |
| Cyp26a1 | cytochrome P450, family 26, subfamily a, polypeptide 1 |
| Pbx4 | pre-B-cell leukemia transcription factor 4 |
| Rabl3 | RAB, member of RAS oncogene family-like 3 |
| Nes | Nestin |
| Fzd2 | frizzled homolog 2 (Drosophila) |
| Scnnlg | sodium channel, nonvoltage-gated 1 gamma |
| Skiv2l2 | Superkiller viralicidic activity 2-like 2 (S. cerevisiae) |
| Mipol1 | mirror-image polydactyly gene 1 homolog (human) |
| | transformation related protein 53 inducible nuclear |
| Trp53inp1 | protein 1 |
| Krtap5-5 | Keratin associated protein 5-5 |
| Prom2 | prominin 2 |

In view of the above, activation of the latent precursor population may be expected with the polypeptides encoded by the nucleic acid molecules set forth in Table 1. The nucleic acid molecules set forth in Table 1 have a nucleotide sequence obtainable from a natural source. Sequence information is publically available in Genbank, EMBL and similar databases. They therefore include naturally occurring normal, naturally occurring mutant, naturally occurring polymorphic alleles, differentially spliced transcripts, splice variants etc. Natural sources include animal cells and tissues, body fluids, tissue culture cells etc.

Polypeptides may be commercially available or prepared in a manner known per se. The nucleic acid molecules set forth in Table 1 can also be engineered using methods accepted in the art so as to alter the gene-encoding sequences for a variety of purposes. These include, but are not limited to, modification of the cloning, processing, and/or expression of the gene product. PCR reassembly of gene fragments and the use of synthetic oligonucleotides allow the engineering of gene nucleotide sequences. For example, oligonucleotide-mediated site-directed mutagenesis can introduce mutations that create new restriction sites, alter glycosylation patterns and produce splice variants etc.

As a result of the degeneracy of the genetic code, a number of nucleic acid sequences encoding the genes referred to in Table 1, some that may have minimal similarity to the nucleic acid sequences of any known and naturally occurring gene, may be produced. In some instances it may be advantageous to produce nucleotide sequences encoding a gene referred to in Table 1 possessing a substantially different codon usage than that of the naturally occurring gene. For example, codons may be selected to increase the rate of expression of the peptide in a particular prokaryotic or eukaryotic host corresponding with the frequency that the host utilizes particular codons. Other reasons to alter the nucleotide sequence of a gene referred to in Table 1 without altering the encoded amino acid sequence include the production of RNA transcripts having more desirable properties, such as a greater half-life, than transcripts produced from the naturally occurring sequence.

In order to prepare purified polypeptides or proteins, host cells may be transfected with a nucleic acid molecule as described above. Typically, said host cells are transfected with an expression vector comprising a nucleic acid molecule as set forth in Table 1. A variety of expression vector/host systems may be utilized to contain and express the sequences. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can also be used to express a protein that is encoded by a specific gene using various expression vectors including plasmid, cosmid and viral systems such as a vaccinia virus expression system.

The nucleic acid molecules can be stably expressed in cell lines to allow long term production of recombinant proteins in mammalian systems. Sequences encoding any one of the genes of the invention can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode a protein may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of protein are needed, vectors which direct high levels of expression may be used such as those containing the T5 or T7 inducible bacteriophage promoter.

Fragments of polypeptides may also be produced by direct peptide synthesis using solid-phase techniques. Automated synthesis may be achieved by using the ABI 431A Peptide Synthesizer (Perkin-Elmer). Various fragments of polypeptide may be synthesized separately and then combined to produce the full length molecule.

Isolation of the neural cell population in which a precursor cell population is activated allows for methods for screening drug candidates for effectiveness in increasing neurogenesis. Screening assays may be performed directly using a culture. Candidate agents may be initially screened for the ability to modulate neurogenesis through its effect on an in vitro culture. For example, in a method which involves contacting the candidate drug and the culture of the present invention, the effect on differentiation and proliferation of the precursor cell population may be observed, but equally the effect on survival, phenotype or function of these cells or their progeny could be observed. An in vivo drug screening or drug discovery process involving engrafting a non-human mammal with an enriched population of neural stem cells is described in U.S. Pat. No. 7,105,150, the contents of which were incorporated herein by reference. The engrafted non-human mammal is useful for drug screening and drug discovery using well known methodology. Methods for screening a candidate agent against a cell culture are described, for example, in U.S. Pat. No. 7,041,438 using methods well known in the art. Assessment of the activity of candidate agents generally involves combining a cell culture with a candidate compound, determining any resultant change, and then correlating the effect of the compound with the observed change.

In addition, differentiated cells arising from the culture of the present invention can be used for tissue reconstitution or regeneration in a human patient in need thereof. The cells are administered in a manner that permits them to graft to the intended tissue site and reconstitute or regenerate the functionally deficient areas. Thus, the in vivo transplantation process comprises implanting members of the cell population generated by the present invention into a mammal once this population has been treated with one or more growth factors to induce differentiation, for example, once they have been induced to differentiate into neurons and/or glia. In vitro proliferation and differentiation of neural stem cells is described, for example, in U.S. Pat. No. 7,115,418, the contents of which are incorporated herein by reference. The growth factors necessary to induce proliferation and/or differentiation are well known to the person skilled in the art and include, but are not limited to, NGF, BDNF, the neurotrophins, CNTF, amphiregulin, FGF-1, FGF-2, EGF, TGFα, TGFβ, PDGF, IGFs and the interleukins.

It will be clearly understood that, although a number of prior art publications are referred to herein, this reference does not constitute an admission that any of these documents forms part of the common general knowledge in the art, in Australia or in any other country.

Throughout this specification and the claims, the words “comprise”, “comprises” and “comprising” are used in a non-exclusive sense, except where the context requires otherwise.

### Example 1

### Activation of Precursor Cell Population

### Primary Neurosphere Cultures

Adult (6-8 week old) male C57/B16 mice were killed by cervical dislocation, their brains immediately removed and the hippocampus, SVZ, olfactory bulb or cortex dissected. The tissue was enzymatically digested with 0.1% trypsin-EDTA (Gibco/Invitrogen, Eugene, Oreg.) for 7 minutes at 37° C., followed by a wash with 0.014% w/v trypsin inhibitor (type I-S from soybean; Sigma-Aldrich Australia, Sydney, Australia) dissolved in Hepes-buffered minimum essential medium (HEM), which consisted of minimum essential medium (Gibco/Invitrogen) supplemented with 16 mM HEPES (Sigma-Aldrich Australia) and 100 units/ml penicillin/streptomycin (Gibco/Invitrogene). The tissue was centrifuged at 100 rcf for 7 minutes, following which the pellet was resuspended in 1 ml neurosphere growth medium, mechanically triturated until smooth, then filtered through a 40 μm cell sieve (Falcon/BD Biosciences Australia, North Ryde, Australia). The neurosphere growth medium consisted of mouse NeuroCult™ NSC Basal Medium plus mouse NeuroCult™ NSC Proliferation Supplements (StemCell Technologies, Vancouver, Canada) with 2% bovine serum albumin (Roche, Basel, Switzerland) and 2 μg/ml heparin (Sigma-Aldrich Australia). The following growth factors were also included: 20 ng/ml purified mouse receptor-grade EGF (BD Biosciences Australia) and 10 ng/ml recombinant bovine FGF-2 (Roche). After centrifugation at 100 rcf for 7 minutes, the cells were plated at a density of approximately one hippocampus, SVZ or cortex or four olfactory bulbs per 96-well plate (Falcon/BD Biosciences Australia) with 200 μl neurosphere medium per well.

The neurosphere growth medium already contained 4.18 mM KCl. Therefore for the depolarization experiments, additional KCl was added to give the final KCl concentration of 20 mM. This level of potassium is around five times that of the normal physiological concentration of potassium, ˜4 mM. Nicardipine (Sigma-Aldrich), when used, was added to a final concentration of 10 μM. BaCl was used as an alternative to KCl in some experiments at a concentration of 1 mM and GABA at 100 μM. Recombinant mouse Wnt3a (R&D Systems) and recombinant mouse prolactin (PRL; R&D Systems) were added instead of KCl in some experiments at concentrations of 1 ng/mL and 2 ng/mL respectively.

Primary hippocampal cells were incubated for 10 days, and SVZ, olfactory bulb or cortical cells for 7 days, in humidified 5% CO₂ to permit neurosphere formation. The primary neurospheres were then counted and collected for passaging or differentiation. Results of the neurosphere counts were expressed as mean±standard error and statistical analysis was performed using a standard t-Test (two sample assuming equal variance).

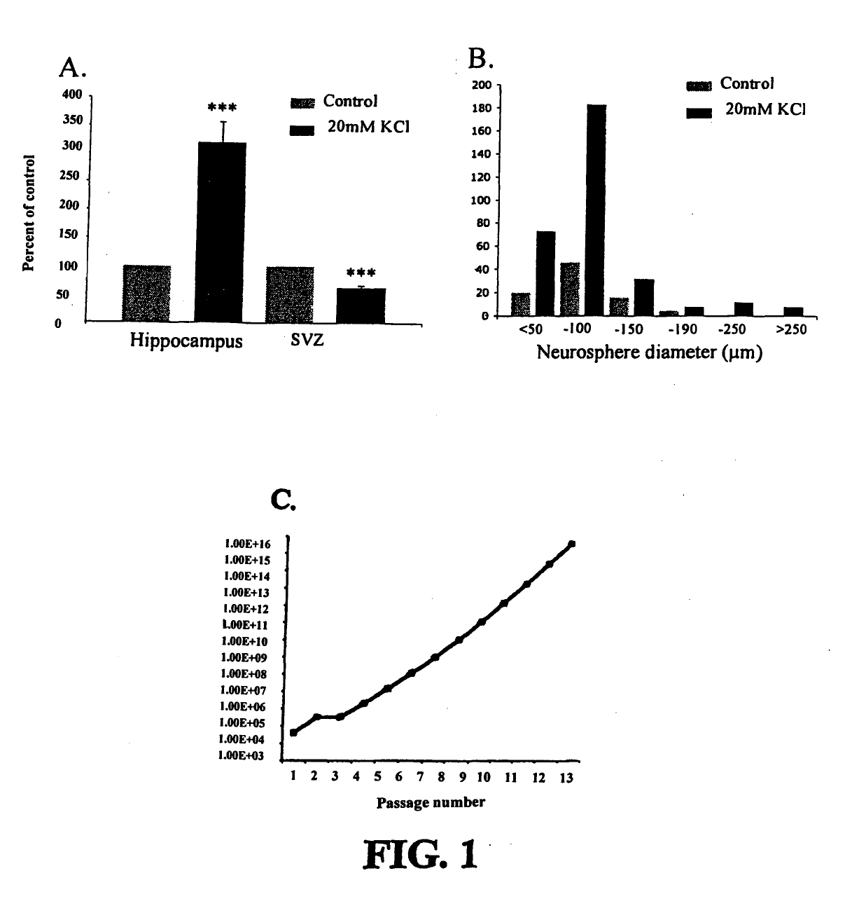

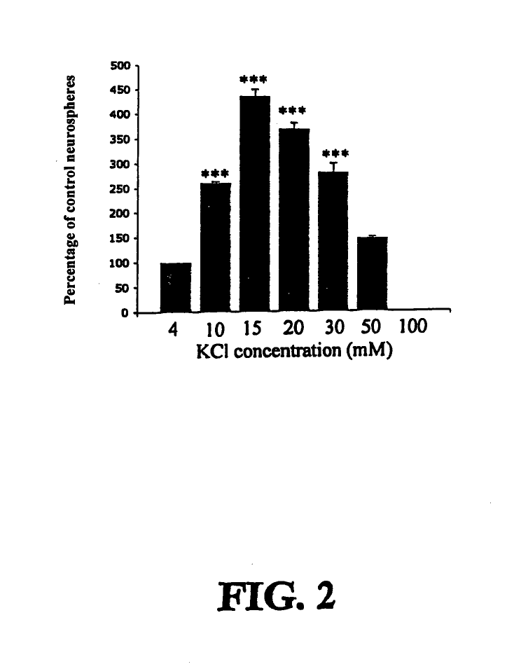

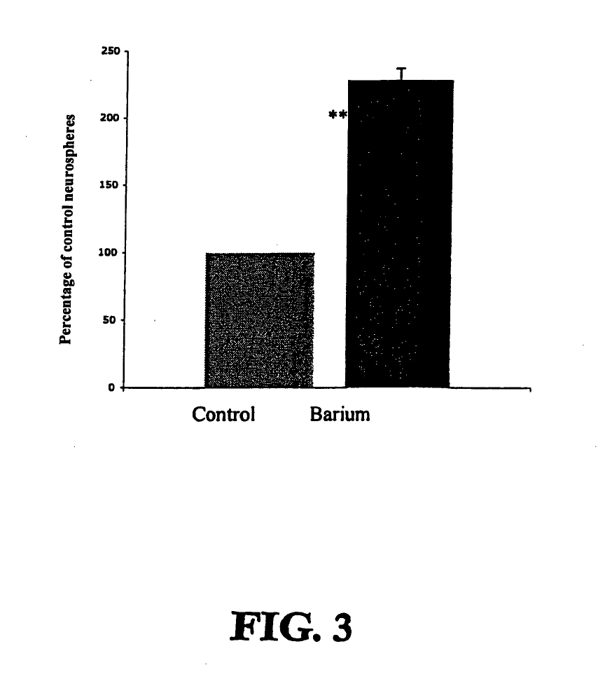

Dissociated, adult mouse hippocampal cells plated in neurosphere-forming media containing 20 mM KCl increased the number of neurospheres generated by over 3-fold compared to those cells plated in control (4 mM) potassium concentrations or non depolarizing control conditions in which an equivalent concentration of sodium was added as an osmotic control (FIG. 1a). A KCl dose response assay further demonstrated that optimal depolarizing level was 15 mM KCl and that a concentration as low as 10 mM and as high as 30 mM will still significantly increase the number of neurospheres generated (FIG. 2). The ability to cause this activation is not restricted to depolarization using KCl as we show that BaCl₂ has similar effect with 1 mM BaCl₂ increasing the numbers of neurospheres over 2-fold (FIG. 3).

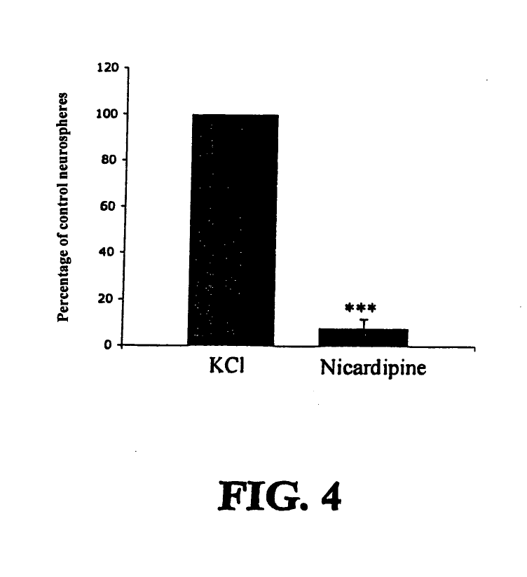

The results show that potassium-induced depolarization stimulates the formation of increased numbers of large neurospheres in the hippocampus (FIGS. 1b and c). The results show further that the depolarization-induced activity of hippocampal precursors is also working through an L-type Ca2+ channel. The L-type Ca2+ antagonist nicardipine almost completely blocked this excitation-induced activation of neurosphere forming cells (FIG. 4).

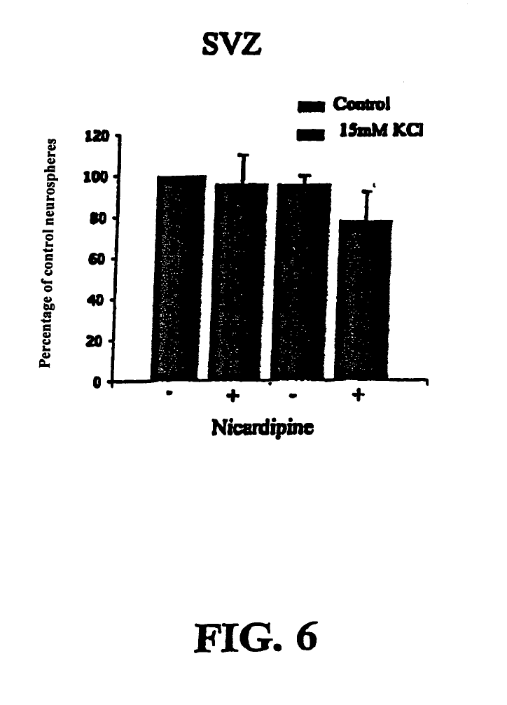

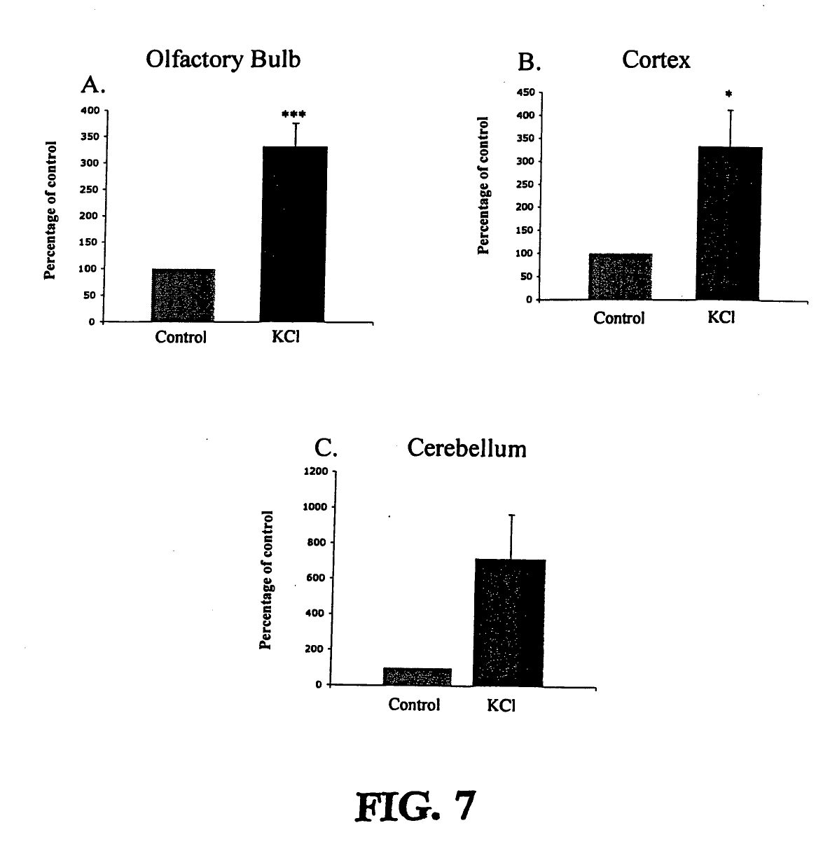

However, potassium-induced depolarization does not increase the number of spheres in the adult SVZ. In fact it decreases neurosphere number by approx 40% (FIG. 6). While not wishing to be bound by theory, it is believed that this indicates that the precursor cell population identified by the present invention is an entirely different population of cells to the one previously identified in the SVZ. Consistent with our results that potassium-induced depolarization does not activate the precursor cells resident in the SVZ, the behavioural paradigms affecting the hippocampus have been shown to have no impact on neurogenesis in the SVZ (Brown et al., 2003). In addition, the L-type Ca2+ channel antagonist nicardipine does not inhibit neurosphere formation in SVZ either in the presence or absence of depolarizing levels of potassium. Neurogenesis in this region can however be increased by olfactory stimulation (Rochefort et al., 2002), further suggesting that different activity rules govern different local circuits. Consistent with this we show that like the hippocampal precursor, the precursor in the olfactory bulb can also be activated under depolarizing conditions. We observe an over 3-fold increase in the number of olfactory bulb neurospheres in the potassium-activated condition compared to control (FIG. 7). In addition there is a significant increase in the size of these activated olfactory-bulb neurospheres (FIG. 7). This population of cells may also be present in other non-proliferating areas. The cortex also showed an increase in neurosphere number in response to depolarization with over 3-fold more neurospheres generated (FIG. 7).

### Hippocampal Neurosphere Passaging

To determine whether the neurospheres generated under depolarizing conditions demonstrated the characteristic stem-cell properties of self-renewal and multipotentiality, primary neurospheres were individually dissociated and replated into media containing no additional potassium. Primary neurospheres from the unstimulated hippocampus were also passaged as single spheres.

Hippocampal neurosphere cultures were initiated by removing 150 μl of the medium from wells containing single neurospheres, treating with 100 μl 0.1% trypsin-EDTA for 2 minutes at room temperature, followed by washing with 100 μl trypsin inhibitor in HEM. The neurospheres were mechanically triturated until dissociated and replated in well plates in 2 ml of complete medium. Neurospheres were passaged every 10 days by centrifuging the neurospheres, removing the medium and incubating in 1 ml of 0.1% trypsin-EDTA for 2 minutes at room temperature. After the addition of an equal volume of trypsin inhibitor, the neurospheres were centrifuged at 100 rcf for 5 minutes and the supernatant removed. Cells were mechanically triturated in 500 μl of complete medium and trypan blue staining was used to evaluate the number of cells, both viable and total number, on a hemocytometer. The passaged cells were then re-plated with complete medium at a density of 1×10⁴ cells/cm² in tissue culture flasks (Nunc, Rochester, N.Y.) or tissue culture plates (Falcon/BD Biosciences) as appropriate.

Although the hippocampal spheres generated under the standard growth conditions never gave rise to cultures that could be passaged continuously (0/113 spheres), a number of the potassium stimulated large primary spheres (23 out of 64) could be expanded in culture for up to 10 passages (FIG. 1c). All twenty three of the KCl stimulated neurospheres capable of long term passage were large with a diameter of >110 μm. Subsequent passages of these hippocampal spheres showed high numbers of neurons when differentiated, indicating they are neurogenic. The primary potassium stimulated neurospheres however, contained mostly astrocytes similar to control neurospheres.

### Example 2

### Microarray Analysis

Hippocampal tissue was dissected and dissociated as described above and incubated either in the presence or absence of 15 mM KCl for 24 hours. Cells were pelleted and total RNA was isolated using the RNeasy mini kit (Qiagen) and quantitated using the Bioanalyzer 2100 (Agilent). aRNA was prepared from 500 ng of RNA using the Message-Amp™-II Biotin Enhanced Kit (Ambion). A total of 15 μg biotinylated aRNA was fragmented and run on a GeneChip mouse Genome 430A 2.0 Array (Affymetrix) in accordance with the manufacturer's instructions.

TABLE 2
The upregulated genes and their change in expression.

| Fold | | |
|---|---|---|
| change | Gene Symbol | Gene name |
| 17.08 | Slc5a1 | solute carrier family 5 (sodium/glucose |
| | | cotransporter), member 1 |
| 14.4 | Wnt3a | wingless-related MMTV integration site 3A |
| 12.8 | Ahnak | AHNAK nucleoprotein (desmoyokin) |
| 12.65 | Tcfap2c | transcription factor AP-2, gamma |
| 12.62 | Setdb1 | SET domain, bifurcated 1 |
| 12.43 | Igf2bp3 | insulin-like growth factor 2 mRNA |
| | | binding protein 3 |
| 12.3 | Iapp | islet amyloid polypeptide |
| 11.82 | Hes2 | hairy and enhancer of split 2 (Drosophila) |
| 11.13 | Gng2 | guanine nucleotide binding protein (G protein), |
| | | gamma 2 subunit |
| 11.05 | Cylc1 | cylicin, basic protein of sperm head cytoskeleton 1 |
| 10.79 | EG434726 /// | ferritin, heavy polypeptide-like 17 /// predicted |
| | EG434727 /// | gene, EG434726 /// predicted gene, EG434727 /// |
| | EG434728 /// | predicted gene, EG434728 |
| | Fthl17 | |
| 10.61 | Igk-V28///I | immunoglobulin kappa chain variable 28 (V28) /// |
| | | immunoglobulin kappa light chain 17-1A |
| 9.84 | Enox2 | ecto-NOX disulfide-thiol exchanger 2 |
| 9.68 | Aurkb | aurora kinase B |
| 9.68 | Zkscan14 | zinc finger with KRAB and SCAN domains 14 |
| 9.6 | Serpina5 | serine (or cysteine) peptidase inhibitor, clade A, |
| | | member 5 |
| 9.59 | Crkrs | Cdc2-related kinase, arginine/serine-rich |
| 9.58 | Ntf3 | neurotrophin 3 |
| 9.43 | Hs1bp3 | HCLS1 binding protein 3 |
| 9.32 | Foxm1 | forkhead box M1 |
| 9.24 | Itgav | integrin alpha V |
| 9.16 | Eif4g1 | eukaryotic translation initiation factor 4, gamma 1 |
| 9.12 | Myb | myeloblastosis oncogene |
| 9.04 | Rabgap1 | RAB GTPase activating protein 1 |
| 8.69 | Rcbtb2 | regulator of chromosome condensation (RCC1) |
| | | and BTB (POZ) domain containing protein 2 |
| 8.53 | D1Ertd83e | DNA segment, Chr 1, ERATO Doi 83, expressed |
| 8.3 | Coll7al | procollagen, type XVII, alpha 1 |
| 8.34 | Pwcr1 | Prader-Willi chromosome region l homolog |
| | | (human) |
| 8.32 | Chrna5 | cholinergic receptor, nicotinic, alpha polypeptide 5 |
| 8.28 | Adh4 | alcohol dehydrogenase 4 (class II), pi polypeptide |
| 8.11 | Serpinb9c | serine (or cysteine) peptidase inhibitor, clade B, |
| | | member 9c |
| 7.97 | Ncapg | on-SMC condensin I complex, subunit G |
| 7.92 | LOC639910 | hypothetical protein LOC639910 |
| 7.89 | Slc22a3 | solute carrier family 22 (organic cation |
| | | transporter), member 3 |
| 7.81 | Lrp6 | low density lipoprotein receptor-related protein 6 |
| 7.72 | Gshl | genomic screened homeo box 1 |
| 7.48 | Dbf4 | DBF4 homolog (S. cerevisiae) |
| 7.43 | Prrg2 | proline-rich Gla (G-carboxyglutamic acid) |
| | | polypeptide 2 |
| 7.43 | LOC669660 /// | PDZand LIM domain 5 /// similar to PDZ and |
| | Pdlim5 | LIM domain protein 5 (Enigma homolog) |
| | | (Enigma- like PDZ and LIM domains protein) |
| 7.21 | Hbb-bhl | hemoglobin Z, beta-like embryonic chain |
| 7.16 | Abcc9 | ATP-binding cassette, sub-family C |
| | | (CFTR/MRP), member 9 |
| 7.09 | Gata6 | GATA binding protein 6 |
| 7.02 | H2-Dl | histocompatibility 2, D region locus 1 |
| 6.77 | Nlrp4c | NLR family, pyrin domain containing 4C |
| 6.76 | Fbxo15 | F-box protein 15 |
| 6.74 | Sim1 | single-minded homolog 1 (Drosophila) |
| 6.73 | Bcl2a1a /// | B-cell leukemia/lymphoma 2 |
| | Bcl2a1b /// | related protein A1a /// B- |
| | Bcl2a1c /// | cell leukemia/lymphoma 2 |
| | Bcl2a1d | related protein A1b /// B- |
| | | cell leukemia/lymphoma 2 related |
| | | protein A1c /// B- |
| | | cell leukemia/lymphoma 2 |
| | | related protein A1d |
| 6.65 | Rab17 | RAB17, member RAS oncogene family |
| 6.36 | Prl | Prolactin |
| 6.35 | Rarres2 | retinoic acid receptor responder |
| | | (tazarotene induced) 2 |
| 6.25 | 2900060N18Rik | RIKEN cDNA 2900060N18 gene |
| 6.22 | Lin7b | lin-7 homolog B (C. elegans) |
| 6.08 | Clgn | Calmegin |
| 6.06 | Ugt8a | UDP galactosyltransferase 8A |
| 6.01 | B3galt1 | UDP-Gal: betaGlcNAc beta 1,3- |
| | | galactosyltransferase, polypeptide 1 |
| 6.01 | Pof1b | premature ovarian failure 1B |
| 5.82 | Atp6v0a1 | ATPase, H+ transporting, lysosomal V0 subunit A1 |
| 5.81 | Bves | blood vessel epicardial substance |
| 5.76 | Itga4 | integrin alpha 4 |
| 5.76 | Iqgap1 | IQ motif containing GTPase activating protein 1 |
| 5.6 | Olfr64///OI | olfactory receptor 64 /// olfactory receptor 66 |
| 5.5 | Tbc1d14 | TBC1 domain family, member 14 |
| 5.39 | Tpt1 | tumor protein, translationally-controlled 1 |
| 5.35 | Rhov | ras homolog gene family, member V |
| 5.33 | Brwdl | bromodomain and WD repeat domain |
| | | containing 1 |
| 5.33 | Krtap15 | keratin associated protein 15 |
| 5.33 | Atf7 | activating transcription factor 7 |
| 5.28 | Scgb1a1 | secretoglobin, family 1A, member 1 |
| | | (uteroglobin) /// cDNA sequence U46068 |
| 5.27 | Gprin1 | G protein-regulated inducer of neurite outgrowth 1 |
| 5.26 | Eras | ES cell-expressed Ras |
| 5.22 | Alx3 | aristaless 3 |
| 5.15 | Avpr1b | arginine vasopressin receptor 1B |
| 5.1 | Cyp26a1 | cytochrome P450, family 26, subfamily a, |
| | | polypeptide 1 |
| 5.02 | Pbx4 | pre-B-cell leukemia transcription factor 4 |
| 4.95 | Rabl3 | RAB, member of RAS oncogene family-like 3 |
| 4.85 | Nes | Nestin |
| 4.81 | Fzd2 | frizzled homolog 2 (Drosophila) |
| 4.74 | Scnnlg | sodium channel, nonvoltage-gated 1 gamma |
| 4.56 | Skiv2l2 | Superkiller viralicidic activity 2-like 2 (S. |
| | | cerevisiae) |
| 4.55 | Mipol1 | mirror-image polydactyly gene 1 homolog |
| | | (human) |
| 4.46 | Trp53inp1 | transformation related protein 53 |
| | | inducible nuclear protein 1 |
| 4.43 | Krtap5-5 | Keratin associated protein 5-5 |
| 4.41 | Prom2 | prominin 2 |

### Example 3

### Characterization of the Latent Precursor Cell Population

DCX-GFP, Emx1-GFP and GFAP-GFP mice are from the Mutant Mouse Regional Resource Center, The Gene Expression Nervous System Atlas BAC transgenic project (Gong et al., 2002).

Brains were collected and processed. GFP^{+ve} cells were separated by fluorescence-activated cell sorting (FACS) using a FACS Vantage cell sorter (BD Biosciences). A wild-type littermate control was used to determine background fluorescence levels. For primary neurosphere cultures the cells were plated at a density of 500 cells/well in 96-well plates (Falcon/BD Biosciences, San Jose, Calif.) with 0.2 ml complete medium per well. Complete medium consisted of mouse NeuroCult™ NSC Basal Medium plus mouse NeuroCult™ NSC Proliferation Supplements (StemCell Technologies,

Vancouver, Canada) with 2% bovine serum albumin (Roche, Basel, Switzerland) and 2 μg/ml heparin (Sigma-Aldrich). The following growth factors were also included: 20 ng/ml purified mouse receptor-grade epidermal growth factor (EGF; BD Biosciences Australia) and 10 ng/ml recombinant bovine basic fibroblast growth factor (FGF-2; Roche). Primary cells were incubated for 7 days in humidified 5% CO₂ to permit neurosphere formation. The primary neurospheres were then counted and collected for passaging or differentiation. Results of the neurosphere counts were expressed as mean+ or −standard error and statistical analysis was performed using a standard t-Test (two sample assuming equal variance).

The latent population was found characterized as:

Emx1 positive: SFU=1/46 Emx1^{+ve}, 1/37 Emx1^{+ve}+KCL 1/10 890 Emx1^{-ve,} 1/10896 Emx1^{-ve}

### +KCL

GFAP positive: SFU=1/295 GFAP^{+ve}, 1/127 GFAP^{+ve}+KCL 1/1869 GFAP^{-ve}, 1/2220 GFAP^{-ve}+KCL
B-tubulin negative
PNA positive

### Example 4

Wnt3a (R&D Systems, Minneapolis) and PRL (R&D Systems, Minneapolis) are able to activate the hippocampal precursor and stem cell populations in vitro

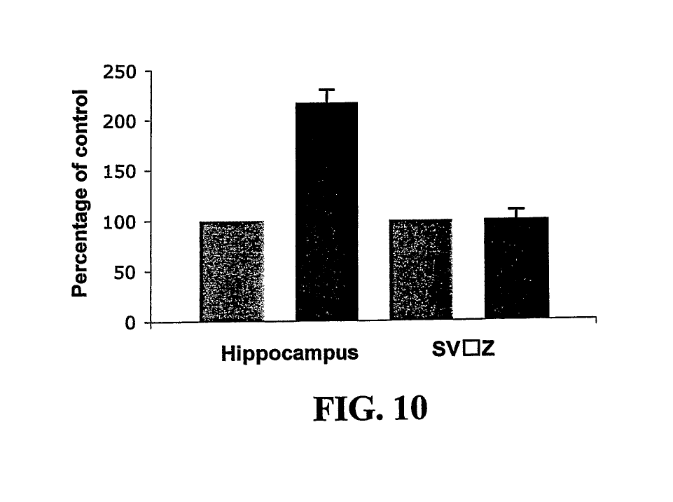

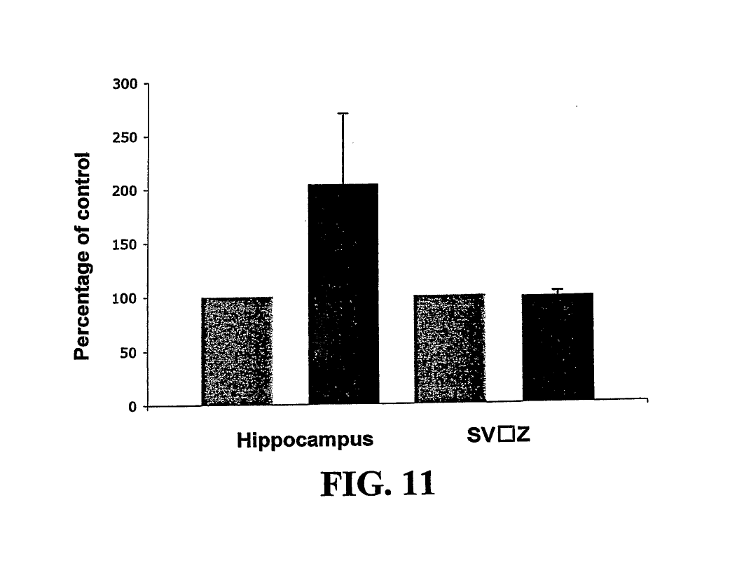

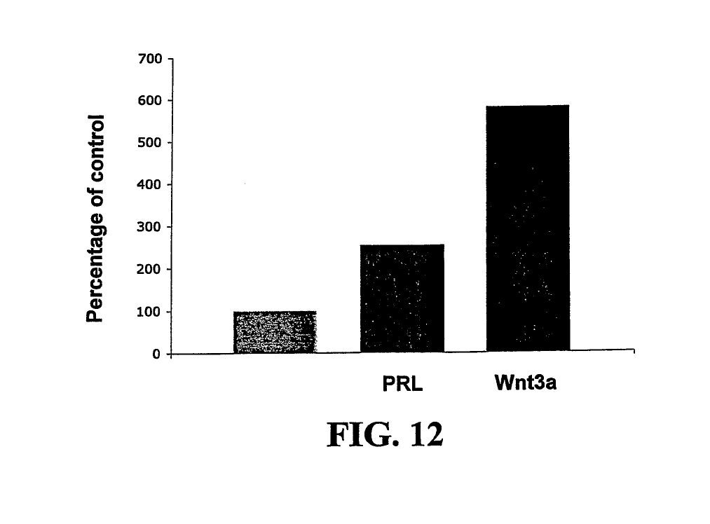

Following depolarization of primary hippocampal cells, microarray analysis revealed a 14.4-fold (P<0.001) up-regulation of Wnt3a and a 6.4-fold (P<0.001) up-regulation of PRL. To test whether these proteins are able to activate the hippocampal precursor and stem cells in vitro primary hippocampal and SVZ cells were exposed to either 1 ng/mL recombinant Wnt3a or 2 ng/mL recombinant PRL. In the presence of either 1 ng/mL Wnt3a or 2 ng/mL recombinant PRL there was a 2-fold increase in the number of hippocampal neurospheres generated (FIG. 11: Wnt3a; 204±66.1% of control, P<0.05, n=3, FIG. 10: PRL; 217±13% of control, P<0.05, n=3). More importantly however, was the fact that both the Wnt3a and PRL treated cultures led to the generation of a number of the very large neurospheres (>250 μm), similar to those observed following KCL depolarization, which could be passaged long-term and upon differentiation gave rise to both neurons and glia. At these concentrations neither protein had any effect on the number of SVZ-derived neurospheres (Wnt3a; 98.5±4.5% of control, n=2; PRL; 100±9% of control, n=2). The effect of Wnt3a and PRL were also tested in the aged (18 month old) hippocampus which revealed over a five-fold and two and a half-fold increase in neurosphere number respectively (FIG. 12: Wnt3a; 580±230% of control, P<0.05, n=4, PRL; 254±23% of control, P<0.001, n=4).

Microarray analysis revealed up-regulation of a number of key proteins in response to KCl-induced depolarization of primary hippocampal cells. Wnts are a family of secreted signalling proteins that act as ligands to activate receptor-mediated signalling cascades in a variety of developmental processes including hippocampal formation, dendritic morphogenesis, axon guidance and synaptic formation. Although Wnt signalling plays a role in many brain functions, the underlying mechanism by which it works to regulate these functions is still relatively unexplored. It has recently been reported that activity-dependant synaptic release of Wnt3a occurs in adult hippocampal neurons in an NMDA-receptor mediated manner (Chen et al., 2006). This study also reported a novel role of Wnt signalling in LTP regulation whereby inhibition of Wnt signalling impairs LTP regulation, and activation potentiates LTP (Chen et al., 2006). In contrast, another study recently demonstrated that Wnt3 is secreted by hippocampal astrocytes and increases adult hippocampal neurogenesis (Lie et al., 2005). Most recently, it has been demonstrated that clinically effective concentrations of lithium directly regulate adult hippocampal neural progenitors through activation of the canonical Wnt pathway thus providing support for a link between mood stabilizers/antidepressants and neurogenesis (Wexler et al., 2007).

Pheromones and their hormonal mediators have been shown to be involved in plasticity at the level of neurogenesis. One such hormone is PRL, which has been shown to stimulate the production of neuronal progenitors in the SVZ of female mice during pregnancy (Shingo et al., 2003). PRL infusion into adult female mice doubled the number of new olfactory interneurons after 4 weeks (Shingo et al., 2003) but had no effect on the number of proliferating cells in the hippocampal dentate gyrus. In addition PRL concentrations of 10 nM and 30 nM in vitro in the presence of EGF increased the numbers of SVZ-derived neurospheres by up to 35% and increased the number of neurons per neurosphere two-fold. In agreement with this earlier study in which 1 nM PRL was shown to have no effect on the number of SVZ-derived neurospheres we observe no effect on neurosphere number in the presence of low concentrations of PRL (2 ng/mL, or 83 pM). Interestingly, although previous studies report that the PRL receptor is not expressed in the dentate gyrus (Mak et al., 2007), we observe a significant increase in the number of hippocampal neurospheres following addition of low doses of PRL in vitro. More importantly however, PRL was able to activate the hippocampal stem cell in vitro leading to the formation of a number of the large stem cell-derived neurospheres that were able to be passaged long-term.

### Example 5

### In Vivo Models

Brain insults such as ischemia and seizure, both of which cause acutely increased local excitation, have shown to cause activity-dependent proliferation in the DG (Arvidsson et al., 2001; Gould et al., 2000; Liu et al., 1998). Example 1 shows that there is a large population of precursor cells which are activated by enhanced synaptic activity. To determine whether these cells can be activated in vivo two models, pilocarpine-induced epileptic seizures and Huntington's disease were investigated. Increased prolonged synaptic activity is seen during seizure therefore, we used this model to see if seizure could activate this population in vivo. Status epilepticus (SE) is defined as continuous seizures lasting for 30 mins or more and results in increased neurogenesis in the DG (Parent et al., 1997).

### Pilocarpine-Induced Seizures

Seizures were induced in 6-8 week old C57BL/6 male mice by the administration of pilocarpine, a muscarinic cholinergic agonist. Thirty minutes prior to pilocarpine administration, the mice were injected with 2.5 mg/kg of the cholinergic antagonist scopolamine methyl nitrate (Sigma-Aldrich) to reduce peripheral cholinergic effects. They then received intraperitoneal injections of 350 mg/kg pilocarpine hydrochloride (Sigma-Aldrich). Seizures were observed in 70% of the mice and of these 35% entered into status epilepticus 78±43 minutes after the injection of pilocarpine. Seizures were stopped 2 hours after status epilepticus onset, with an intraperitoneal injection of 5 mg/kg diazepam (Pamlin, Parnell Laboratories Sydney, Australia). Mice that did not enter status epilepticus also received diazepam and were used as controls.

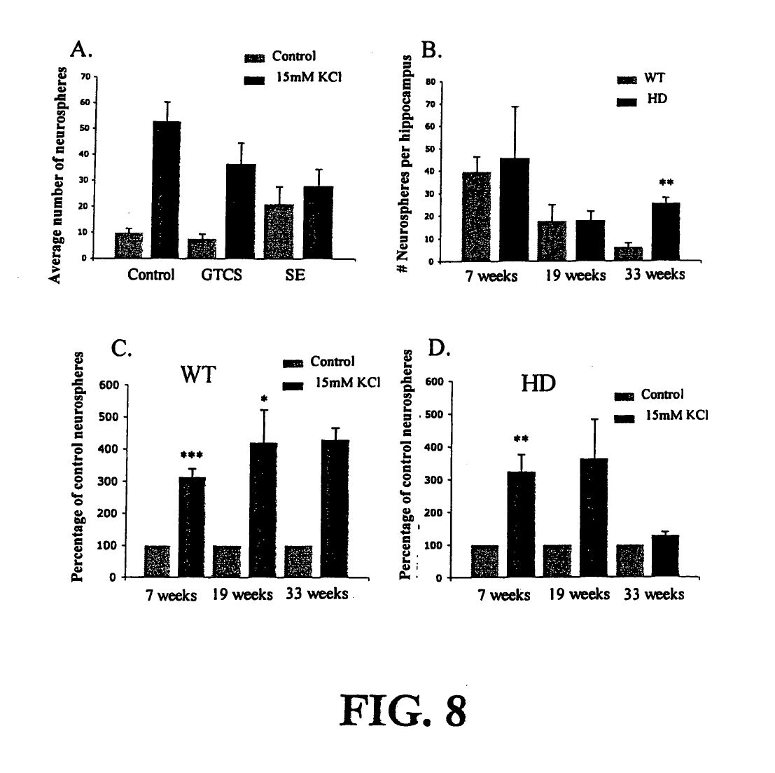

Following SE there is considerable hippocampal damage (Cilio et al., 2003) and a transient increase in proliferation lasting 2-3 weeks (Parent et al., 1997) however, the majority of the new neurons do not survive beyond 4 weeks (Ekdahl et al., 2001). Interestingly, having a small number of seizures GTCS does not cause this significant increase in neurogenesis. Consistent with this increased synaptic activity, we show that mice which underwent SE for at least 2 hours and sacrificed after 48 hours showed a significant increase in the numbers of hippocampal neurospheres compared to control mice (FIG. 8). Interestingly, mice that suffered sporadic GTCS had no increase in hippocampal neurosphere numbers. Most importantly the precursors from mice that underwent SE were activated completely and addition of depolarizing levels of potassium had no further stimulatory effect indicating it is the same population of precursors (FIG. 8). In addition we observed that extended depolarization, between 24 and 48 hours exposure to high level of KCl is required for the stimulation to occur in WT hippocampus confirming the need for prolonged depolarization.

### Huntington's Disease Mice

R6/1 (B6CBA-TgN(HDexon1)61Gpb/Pan) mice were originally obtained from Jackson Laboratory (Bar Harbor, Me.) and maintained by backcrossing to CBB6F1 females. For the neurosphere assays we used HD exon 1 carriers (R6/1) and aged matched wild-type littermates. Huntington's Disease is a neurodegenerative disorder that affects the striatum and cortex as well as hippocampus (Murphy et al., 2000). Previous studies have shown that older symptomatic R6/1 transgenic mice have a significant decrease in cell proliferation in the hippocampus compared to wild-type mice whereas younger pre-symptomatic R6/1 and WT mice had similar levels of proliferation (Lazic et al., 2004). In addition, neurogenesis failed to upregulate in the DG of R6/2 mice in response to seizures suggesting these mice have a lower capacity to mobilise neurogenesis in response to injury (Phillips et al., 2005). We observed a decrease in the total number of hippocampal neurospheres with age in WT mice that was not observed in HD mice. Older symptomatic HD mice (33 weeks old) have significantly more neurospheres than the 33 week old WT mice, and a similar number of neurospheres to both the 19 week old WT and HD mice (FIG. 8). Consistent with the seizure model we found that the 33 week HD hippocampal precursors were activated completely and could not be further increased by potassium depolarization in vitro (FIG. 8).

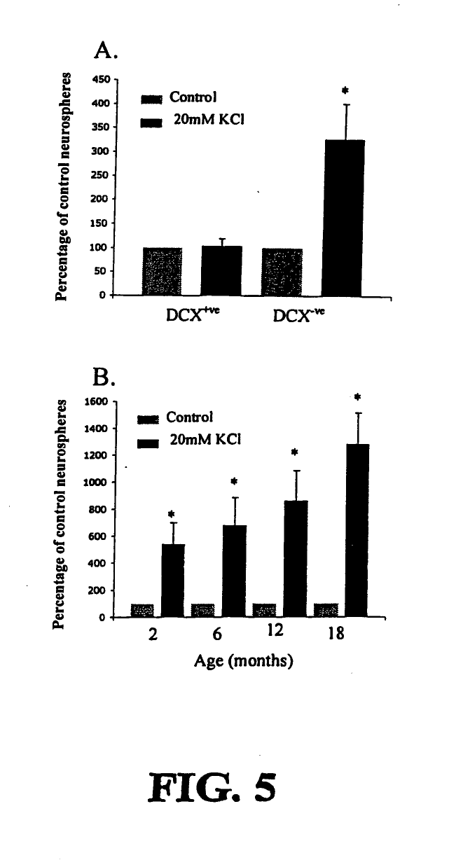

An age related decrease has been shown to occur in the number of restricted progenitors but not in the number of neural stem cells in the subependyma (Tropepe et al, 1997), however an age related decrease of about 10-17% in cell proliferation below adult levels been shown (Kuhn et al., 1996; Seki and Ari, 1995). Conversely, no age-related attenuation of proliferation was observed in the lateral ventricle wall (Kuhn et al., 1996). We show that there is a significant decrease in the number of neurosphere-forming cells in the hippocampus as it ages (FIG. 5b). However, the percentage increase in cells stimulated by the depolarizing conditions increases with age. The greatest increase was observed in the oldest animals we tested with the 18 month old hippocampus demonstrating an over 12-fold increase in the number of neurospheres formed in the stimulated compared to control conditions. Consistent with mechanisms of regulation of neurogenesis, animals with low neurogenesis, for example the DBA mouse strain, have a smaller population and show a decreased percentage of activation.

### PRL and PRL Receptor Knock-Out Mice

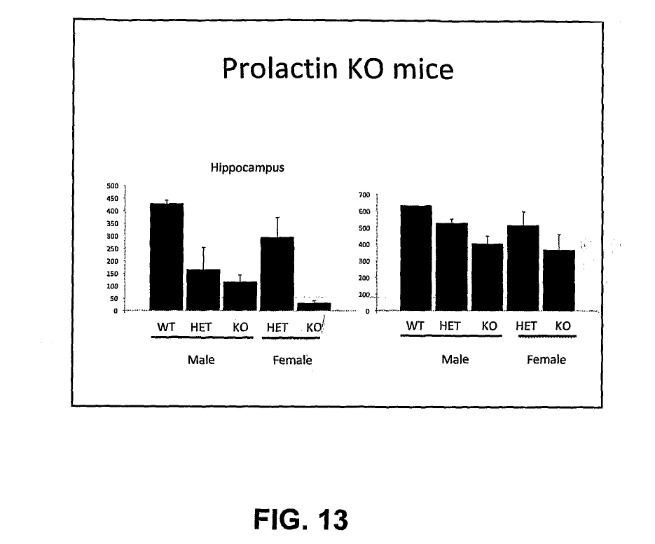

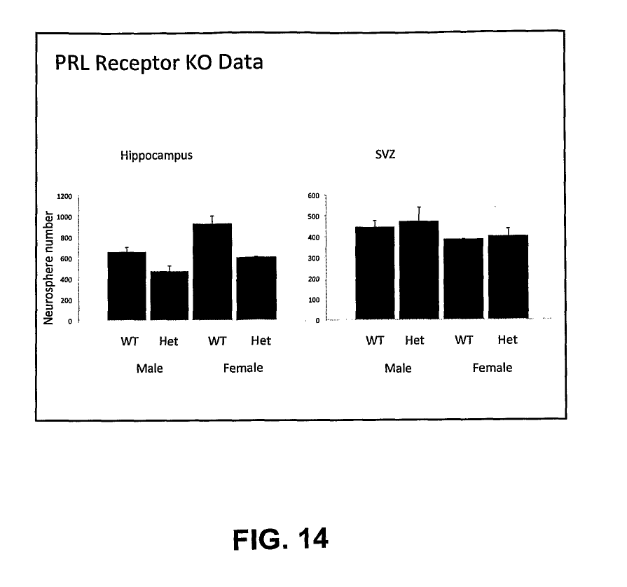

Prolactin knock-out and prolactin receptor knock-out mice were prepared as described by Horseman et al (1997) and Ormandy et al (1997), respectively. It was observed (FIG. 13) that less neurospheres are present in a mouse heterozygous for an inactivating mutation in the prolactin receptor than in the wild-type. It was also observed (FIG. 14) that a prolactin knock-out mouse produced much fewer neurospheres than wild-type or a heterozygote, which itself produced less than the wild-type. Accordingly it is concluded that prolactin acting through the prolactin receptor influences neurosphere formation.

### INDUSTRIAL APPLICABILITY

The ability to induce proliferation and/or differentiation in a neural cell population pharmacologically in vivo has important clinical implications, for example, in repopulation of a damaged hippocampus after brain injuries such as stroke or ischemia leading to some degree of long-range connections and improved functional recovery.

### REFERENCES

The contents of the following documents cited within the preceding description are incorporated herein by reference.
C. M. Batista, T. E. Kippin, S. Willaime-Morawek, M. K. Shimabukuro, W. Akamatsu, D. van der Kooy, J. Neurosci. 26, 13411 (2006).
J. Brown, C. M. Cooper-Kuhn, G. Kempermann, H. van Praag, J. Winkler, F. H. Gage, H. G. Kuhn, Eur. J. Neurosci. 17, 2042 (2003).
N. D. Bull, P. F. Bartlett, J. Neurosci. 25, 10815 (2005).
K. Deisseroth, S. Singla, H. Toda, M. Monje, T. D. Palmer, R. C. Malenka, Neuron 42, 535 (2004).
B. S. Mc Ewen, Ann. N Y Acad. Sci. 746, 134 (1994).
S. A. Goldman, F. Nottebohm, Proc. Natl. Acad. Sci. USA 80, 2390 (1983).
Gong S, Yang X, Li C, Heintz N (2002) Homologous recombination based modification of bacterial artificial chromosomes (BACs) using novel shuttle vectors containing the R6Kγ origin of replication. Genome Res 12:1992-1998.
Horseman N, Zhao W, Montecino-Rodriquez E, Tanaka M, Nakashima K, Engle S, Smith F, Markoff E, Dorshkind K 1997 Defective mammopoiesis, but normal hematopoiesis, in mice with targeted disruption of the prolactin gene. EMBO J 16: 101-110
L. Mangiarini, K. Sathasivam, M. Seller, B. Cozens, A. Harper, C. Hetherington, M. Lawton, Y. Trottier, H. Lehrach, S. W. Davies, G. P. Bates, Cell 87, 493 (1996).
Ormandy C, Camus A, Barra J, Damotte D, Lucas B, Buteau H, Edery M, Brousse N, Babinet C, Binart N, Kelly P A 1997b Null mutation of the prolactin receptor gene produces multiple reproductive defects in the mouse. Genes Dev 11: 167.
J. M. Parent, T. W. Yu, R. T. Leibowitz, D. H. Geschwind, R. S. Sloviter, D. H. Lowenstein, J. Neurosci. 17, 3727 (1997).
V. Ramirez-Amaya, D. F. Marrone, F. H. Gage, P. F. Worley, C. A. Barnes, J. Neurosci. 26, 12237 (2006).
Rickert, R C. et al. (1997). Nucleic Acids Res. 25: 1317-1318.
L. Santarelli, M. Saxe, C. Gross, A. Surget, F. Battaglia, S. Dulawa, N. Weisstaub, J. Lee, R. Durman, O. Arancio et al., Science 301, 805 (2003).
R. M. Seaberg, D. van der Kooy, J. Neurosci. 22, 1784 (2002).
Schwenk, F. et al. (1995). Nucleic Acids Res. 23: 5080-5081.
T. J. Shors, G. Miesegaes, A. Beylin, M. Zhao, T. Rydel, E. Gould, Nature 410, 372 (2001).
H. van Praag, B. R. Christie, T. J. Sejnowski, F. H. Gage, Proc. Natl. Acad. Sci. USA 96, 13427 (1999).

1-8. (canceled)

9. A method of establishing a neural cell population enriched for latent neural precursor cells, comprising:
(1) providing a hippocampal cell population comprising a latent neural precursor cell;
(2) introducing the hippocampal cell population to a neurosphere-forming culture medium;
(3) introducing prolactin or Wnt3a to the culture medium so as to activate the latent neural precursor cell population; and
(4) selecting cells which demonstrate the property of self-renewal and multipotency.

10. A method as claimed in claim 9 wherein prolactin is introduced to the culture medium.

11. A method as claimed in claim 9 wherein Wnt3a is introduced to the culture medium.

12. A method as claimed in claim 9 wherein cells from large neurospheres are selected.

13. A method as claimed in claim 12 wherein cells from neurospheres larger in diameter than 110 mm are selected.

14. A method as claimed in claim 12 wherein cells from neurospheres larger in diameter than 250 mm are selected.

15-22. (canceled)
